(19) United States
(12) Patent Application Publication
(10) Pub. No.: US 2009/0149766 A1
(43) Pub. Date: Jun. 11, 2009

(54) CORONARY VEIN HEMODYNAMIC SENSOR

(76) Inventors: Allan C. Shuros, (US); Lili Liu, (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/256,203

(22) Filed: Oct. 22, 2008

Related U.S. Application Data
(60) Provisional application No. 61/012,872, filed on Dec. 11, 2007.

(51) Int. Cl. A61B 5/02

(52) U.S. Cl. 600/508

(57) ABSTRACT

A system and method for estimating a hemodynamic performance parameter value of a patient's heart. The system includes a pulse generator and a medical electrical lead implanted partially within a coronary vein of heart. The lead includes at least one sensor located within the coronary vein configured to generate a signal indicative of at least one dimensional parameter of the coronary vein. Changes in the dimensional parameter during one or more cardiac cycles are measured. The hemodynamic performance parameter is estimated based on the change in the dimensional parameter of the coronary vein.

### CROSS REFERENCE TO RELATED APPLICATION

This application claims the benefit under 35 U.S.C. §119 of U.S. Provisional Application No. 61/012,872, filed Dec. 11, 2007, titled “CORONARY VEIN HEMODYNAMIC SENSOR,” the entirety of which is hereby incorporated by reference.

### TECHNICAL FIELD

The present invention relates to a method and system for estimating and monitoring cardiac hemodynamic performance, and in particular, for estimating hemodynamic performance utilizing one or more sensing elements coupled to a lead implanted in a coronary vein.

### BACKGROUND

Various measures have been identified for estimating and evaluating reduced cardiac function. Such measures include left ventricular pressure (LVP), which can be particularly useful in estimating and evaluating cardiac hemodynamic performance. Direct measurement of LVP requires locating one or more pressure sensors directly in the left ventricle, which can be both technically and clinically challenging. There is thus a need for improved systems and methods for estimating and evaluating cardiac hemodynamic performance.

### SUMMARY

The present invention, in one embodiment, is a method of estimating cardiac hemodynamic performance. The method comprises implanting a medical electrical lead partially within a coronary vein of a patient's heart, the lead including at least one sensing element positioned within the coronary vein. The method further comprises generating a signal indicative of at least one dimensional parameter of the coronary vein using the sensing element, and estimating a hemodynamic performance parameter as a function of changes in the signal during each of a plurality of cardiac cycles.

In another embodiment, the present invention is a method of estimating cardiac hemodynamic performance. The method comprises estimating a change in a dimensional parameter of a coronary vein during a cardiac cycle, and thereby estimating a hemodynamic performance parameter based on the change in the dimensional parameter during each of a plurality of cardiac cycles.

In still another embodiment, the present invention is a method of treating a heart of a patient. The method comprises generating a signal indicative of a dimensional parameter of a coronary vein, and estimating a hemodynamic performance parameter as a function of changes in the signal during each of a plurality of cardiac cycles. The method further comprises selecting a therapeutic response based at least in part on the cardiac hemodynamic performance parameter.

In yet another embodiment, the present invention is a system for estimating hemodynamic performance of a heart. The system comprises a medical electrical lead configured to be partially implanted in a coronary vein of the heart. The lead includes a sensing element configured to generate a signal indicative of a dimensional parameter of the coronary vein. The system further comprises a processor operatively coupled to the lead configured to estimate a hemodynamic performance parameter based on changes in the signal during each of a plurality of cardiac cycles.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a cardiac rhythm management system including a pulse generator and a lead according to one embodiment of the present invention.

FIG. 2 is a schematic view of a distal end portion of the lead of FIG. 1 positioned in a coronary vein according to one embodiment of the present invention.

FIG. 3 is a schematic view of a distal end portion of the lead of FIG. 1 including an occluding member and positioned within a coronary vein according to another embodiment of the present invention.

FIGS. 4A and 4B are schematic views of the distal end portion of the lead of FIG. 1 including an alternative occluding member according to another embodiment of the present invention.

FIG. 5 is a flow chart illustrating an exemplary method of estimating cardiac hemodynamic performance utilizing the cardiac rhythm management system of FIG. 1 according to one embodiment of the present invention.

FIG. 6 is a schematic partial cut-away view of a lead for use with the cardiac rhythm management system of FIG. 1 positioned in the coronary vein according to another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

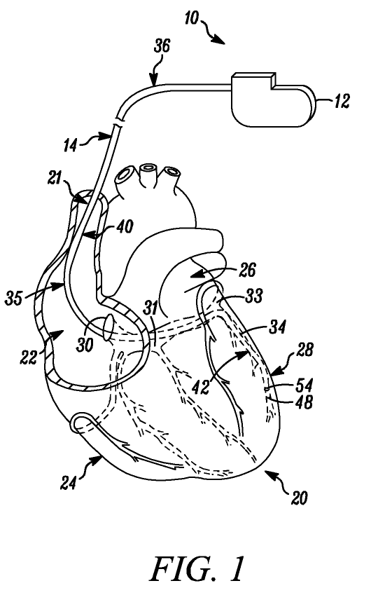

FIG. 1 is a schematic view of a cardiac rhythm management system 10 including a pulse generator 12 coupled to a lead 14 deployed in coronary vein of a patient's heart 20 from a superior vena cava 21. As is known in the art, the pulse generator 12 is typically implanted subcutaneously at an implantation location in the patient's chest or abdomen. As shown, the heart 20 includes a right atrium 22 and a right ventricle 24, a left atrium 26 and a left ventricle 28, a coronary sinus ostium 30 in the right atrium 22, a coronary sinus 31, and various coronary veins including a great cardiac vein 33 and an exemplary branch coronary vein 34.

As shown in FIG. 1, the lead 14 includes an elongate body 35 defining a proximal region 36 and a distal region 40. The distal region 40 has a distal end portion 42 terminating in a distal tip 48. In the embodiment illustrated in FIG. 1, the distal region 40 is guided through the superior vena cava 21, the right atrium 22, the coronary sinus ostium 30, and the coronary sinus 31, and into the coronary vein 34, with the distal end portion 42 positioned therein. The illustrated position of the lead 14 may be used, for example, for sensing physiologic parameters and delivering a pacing and/or defibrillation stimulus to the left side of the heart 20. The lead 14 may also be partially deployed in other coronary veins such as the great cardiac vein 33 or other branch vessels for providing therapy to the left side (or other portions) of the heart 20.

As further shown in FIG. 1, the lead 14 includes at least one sensing element 54 in the distal end portion 42, which in the implanted position of FIG. 1 is also located in the coronary vein 34. As will be explained in greater detail below, in the illustrated embodiment, the sensing element 54 is configured to sense one or more dimensional parameters, e.g., volume, diameter, circumference, of the coronary vein 34. As such, the sensing element 54 can be used to generate signals indicative of such dimensional parameters. Furthermore, when processed in the pulse generator 12 or elsewhere, these signals may be utilized to measure changes in the dimensional parameters during the cardiac cycle, and such measured changes can be utilized to estimate hemodynamic performance parameters, control patient therapy, and the like.

For example, the coronary vein 34 will undergo changes in internal volume during a cardiac cycle. In particular, the internal volume V of the coronary vein 34 will generally decrease as the cardiac chambers fill during diastole. Similarly, the internal volume V of the coronary vein 34 tends to increase as the internal pressure and blood flow within the coronary vein 34 increase during systole. Thus, by comparing the systolic internal volume V_{sys} and the diastolic internal volume V_{dia} of the coronary vein 34 during a cardiac cycle, the change in internal volume ΔV of the coronary vein 34 during the cardiac cycle can be determined and monitored.

The change in coronary venous volume ΔV during the cardiac cycle will correlate well with changes in LVP during the cardiac cycle, to substantially the same extent to which coronary venous pressure correlates with LVP, as disclosed in, for example, U.S. Pat. No. 6,666,826 to Salo, et al., which is incorporated herein by reference in its entirety. As is generally known, LVP is an important measure for evaluating the hemodynamic performance of the heart. Thus, the various embodiments of the present invention configured for sensing and measuring changes in coronary vein internal volume can facilitate similar measurement and evaluation of hemodynamic performance without requiring direct measurement of LVP or coronary venous pressure. In other words, according to the various embodiments of the present invention, the coronary venous internal volume changes during the cardiac cycle can be utilized to derive any of the hemodynamic performance parameters that can be derived by measuring coronary venous pressure and/or LVP.

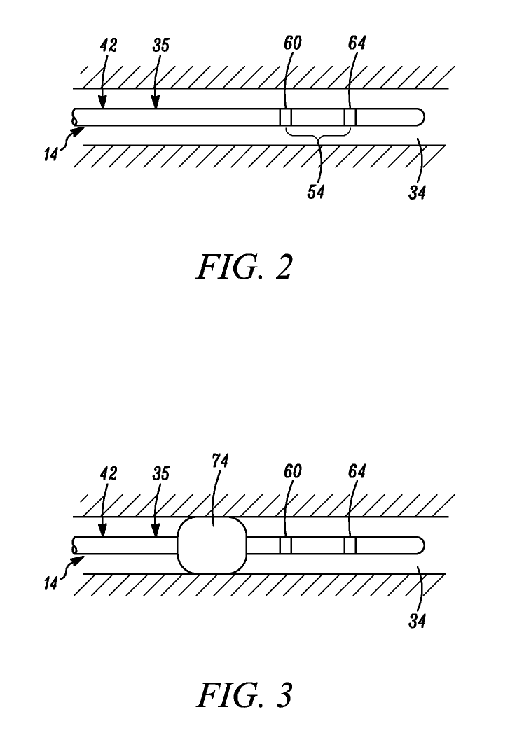

FIG. 2 is a schematic view of the distal end portion 42 of the lead 14 positioned in the coronary vein 34. As shown in FIG. 2, in the illustrated embodiment, the sensing element 54 includes a pair of longitudinally-spaced electrodes 60, 64 coupled to the lead body 35. As will be appreciated, the electrodes 60, 64 are electrically isolated from one another, and are each electrically coupled to a separate contact at the proximal end of the lead 14. The electrodes 60, 64 are further electrically coupled to a processor and/or circuitry within the pulse generator 12 (see FIG. 1) for facilitating sensing and measuring dimensional parameters of the coronary vein 34, for sensing cardiac electrical activity, and/or providing an electrical stimulus to the cardiac tissue.

In the illustrated embodiment, the electrodes 60, 64 are utilized to sense the localized impedance within the portion of the coronary vein 34 in which they are implanted. In one embodiment, an electrical current is sent from the pulse generator 12 (see FIG. 1) to one of the electrodes 60 or 64, and the resulting electric field is sensed by the other electrode 64 or 60, thus generating a signal from which the pulse generator can measure the localized impedance within the coronary vein 34 adjacent the electrodes 60, 64. Localized impedance within the coronary vein 34 will change generally proportionately with changes in localized internal volume of the coronary vein 34 during the cardiac cycle. Thus, by measuring impedance within the coronary vein 34 across the electrodes 60, 64 substantially continuously during the cardiac cycle utilizing the lead 14, changes in internal volume within the coronary vein 34 during the cardiac cycle can be estimated. Additionally, the changes in coronary vein volume during the cardiac cycle, and in particular, the difference between the systolic internal volume V_{sys} and the diastolic internal volume V_{dia} of the coronary vein 34 can be used to estimate and monitor cardiac hemodynamic performance.

The lead 14, including the electrodes 60, 64, can be of any design and construction, whether now known or later developed, suitable for leads for use in bi-ventricular pacing and/or cardiac resynchronization therapy. Thus, the particular configuration of the lead 14 can be varied to satisfy the needs of the particular patient anatomy. For example, the longitudinal spacing between the electrodes 60, 64 may be varied among various leads 14 to provide the optimal sensitivity for impedance and volume measurements. In various embodiments, the electrodes 60, 64 may be positioned approximately 0.5 to 2 centimeters apart. In other embodiments, the electrodes 60, 64 may have different spacing therebetween.

In various embodiments, the electrodes 60, 64 may further be configured as pacing and/or sensing electrodes, as are known in the art. Additionally, in various embodiments, the lead 14 can include additional electrodes for impedance/volume measurements and/or for pacing and sensing. As will be appreciated, selection of the particular functionality of the electrodes can be accomplished, in various embodiments, by the pulse generator 12 (see FIG. 1). For example, in various embodiments, the lead 14 may include more than two electrodes configured for impedance sensing, and the pulse generator 12 can be programmed to select the particular pair of electrodes used for measuring impedance within the coronary vein 34. Still other combinations of electrode functionality will become apparent to those skilled in the art based on the foregoing.

The lead 14 is overall sized and shaped to provide the desired functionality according to the particular needs of the patient. In various embodiments, the lead 14 may be configured in substantially the same manner as conventional coronary venous leads for cardiac resynchronization therapy, bi-ventricular pacing, and the like, which may be modified as described herein to facilitate sensing and measuring coronary vein dimensional parameters. In various embodiments, the lead 14 may be sized and configured to partially occlude a portion of the coronary vein 34 to effectively form a chamber in which the electrodes 60, 64 are positioned when implanted. In some circumstances, complete or partial occlusion of the coronary vein can result in increased sensitivity in the coronary vein volume measurements. In other embodiments, however, the size and configuration of the lead 14 are selected such that it does not significantly occlude the coronary vein 34.

FIG. 3 is a schematic view of the distal end portion 42 of the lead 14 positioned in the coronary vein 34 according to another embodiment of the present invention. In the embodiment illustrated in FIG. 3, the lead 14 is configured to be substantially similar to that shown in FIG. 2, with the addition of an expandable occlusion member 74 coupled to exterior of the lead body 35 proximal to the electrodes 60, 64. The occlusion member 74 is provided in the form of an inflatable balloon which, when inflated as shown in FIG. 3, extends radially outward from the lead body 35 to contact and engage the interior wall of the coronary vein 34. In this manner, the occlusion member 74 operates when inflated to substantially or completely occlude the coronary vein 34 proximal to the electrodes 60, 64, thereby providing a substantially isolated portion or chamber within the coronary vein 34 in which volumetric changes are sensed and measured according to the embodiments of the present invention described herein.

The particular configuration of the occlusion member 74 is not critical. That is, the occlusion member 74 can be configured in any manner suitable for providing a flexible, inflatable structure that can substantially sealingly engage the walls of the coronary vein 34 so as to provide the desired occlusive effect. In various embodiments, the occlusion member 74 may be made from any flexible, biocompatible polymer material such as, without limitation, silicone rubber, polyurethane, and the like. As will be readily appreciated by those skilled in the art, the occlusion member 74 may be inflated using any suitable compressible or non-compressible biocompatible fluid, e.g., air, nitrogen, saline, etc., such as are used in conjunction with balloon catheters and the like. Such fluids can be introduced into the interior of the occlusion member 74 via an inflation lumen (not shown) accessible to the clinician at or near the proximal end of the lead 14. In short, the specific configuration of the occlusion member 74 and the means for inflating it are not critical.

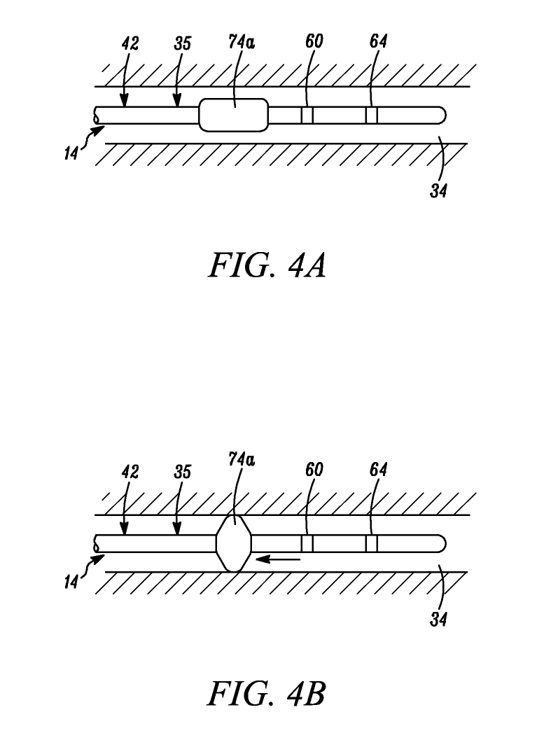

FIGS. 4A and 4B are schematic views of the distal end portion 42 of the lead 14 according to another embodiment of the present invention. The lead 14 illustrated in FIGS. 4A and 4B is substantially similar to that illustrated above, and so identical features are given the same reference numbers. In the illustrated embodiment, the lead 14 includes an alternative occlusion member 74a coupled to the exterior of the lead body 35 in the distal end portion 42 proximal to the electrodes 60, 64. The occlusion member 74a is a radially self-expanding structure, which when in an un-deployed and un-expanded state (FIG. 4A), has a relatively low profile and does not extend significantly beyond the outer surface of the lead body 35. This un-expanded configuration facilitates delivery of the lead 14 to its desired implantation site without excessive interference with the coronary vasculature and/or any delivery catheters, guides or sheaths through which the lead 14 may be delivered.

As shown in FIG. 4B, in an expanded or deployed state, the occlusion member 74a extends radially outward with respect to the lead body 35 so as to contact and engage the coronary vein walls, and thereby to substantially occlude the coronary vein 34 proximal to the electrodes 60, 64. As with the embodiment illustrated in FIG. 3, the occlusion member 74a when deployed as shown in FIG. 4B thus defines a substantially isolated portion or chamber within the coronary vein 34 in which volumetric changes are sensed and measured according to the embodiments of the present invention described herein.

The occlusion member 74a may, in one embodiment, be a self-expanding structure that is maintained in the radially collapsed configuration shown in FIG. 4A during delivery. In such an embodiment, the occlusion member 74a is allowed to assume its pre-biased expanded form when the lead is positioned as desired by the clinician. For example, in one embodiment, the occlusion member 74a may be a stent-like structure covered by a suitable flexible material, and it and the lead 14 in general may be configured such that the occlusion member 74a can be maintained in an elongated, radially collapsed configuration of FIG. 4A using a stylet or other delivery tool. In such an embodiment, removal of the stylet will allow the occlusion member 74a to expand radially to its deployed configuration as illustrated in FIG. 4B.

In various other embodiments, the lead 14 may include other types of occlusion elements. For example, the lead 14 may include any of the occlusion elements described in U.S. Pat. No. 6,666,826 to Salo, et al., which is incorporated herein by reference in its entirety. It is emphasized, however, that in various other embodiments, such as that illustrated in FIG. 2, the lead 14 does not include an occlusion element.

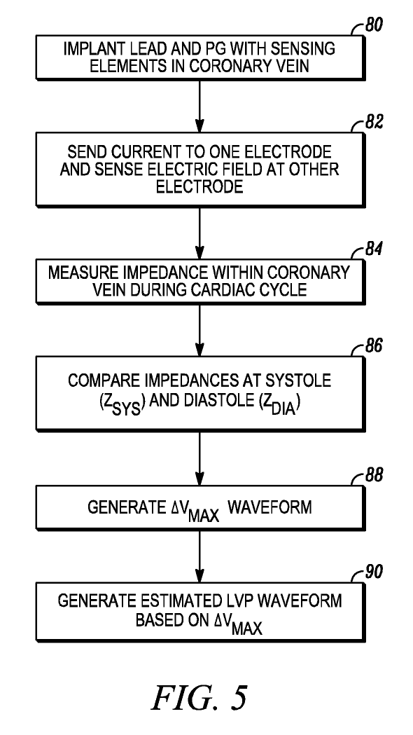

FIG. 5 is a flow chart illustrating a method of estimating cardiac hemodynamic performance utilizing the cardiac rhythm management 10 according to one embodiment of the present invention. As shown in FIG. 5, the cardiac rhythm management system, in particular, the pulse generator 12 and the lead 14 are implanted in the patient according to methods known in the art. (Block 80) Accordingly, as part of this process, the lead 14 is positioned as desired by the clinician, with the electrodes 60, 64 positioned in the selected coronary vein 34. In some embodiments, this may also include inflating or otherwise expanding an occlusion member on the lead 14, e.g., the occlusion members 74 and 74a described above, to completely or partially occlude the coronary vein 34. As explained above, however, in various embodiments, the lead 14 itself may provide any desired occlusive effect. In still other embodiments, as discussed above, the coronary vein 34 need not be occluded.

Once the pulse generator 12 and the lead 14 are implanted and calibrated, coronary vein volume changes can be measured. In one embodiment, one of the electrodes 60 or 64 is configured within the pulse generator 12 to function as a stimulation electrode, and the other electrode 64 or 60 is configured to function as a sensing electrode. Thus, the pulse generator 12 sends an electric current to the stimulation electrode, and the pulse generator 12 measures the magnitude of the corresponding electrical field sensed by the sensing electrode. (Block 82) From the magnitudes of the transmitted current and the sensed electric field, the processor within the pulse generator 12 then measures the impedance Z within the coronary vein 34. (Block 84)

Next, as shown in FIG. 5, the desired systolic and diastolic impedances Z_{sys} and Z_{dia} within each cardiac cycle to be monitored are determined and compared. (Block 86) From this comparison, the desired hemodynamic performance parameter values can be derived. In the illustrated embodiment, for example, a waveform indicative of the maximum change in coronary venous internal volume ΔVₘₐₓ over a plurality of cardiac cycles is generated. (Block 88) From there, as further shown, an estimated LVP waveform can be generated based on the ΔVₘₐₓ waveform, and any number of hemodynamic performance parameters can be derived therefrom. (Block 90) By way of example only, such hemodynamic performance measures may include coronary venous perfusion and coronary artery perfusion.

In various embodiments, the pulse generator 12 may be configured to measure impedance Z substantially continuously throughout the cardiac cycle. In such embodiments, the pulse generator 12 may estimate ΔVₘₐₓ by subtracting maximum and minimum measured impedance values. In other embodiments, the pulse generator 12 may be programmed to identify Z_{sys} and Z_{dia} corresponding to the maximum and minimum coronary venous internal volumes based on ECG data derived conventionally from sensed cardiac electrical activity. In such embodiments, the Z_{sys} value corresponding to the maximum coronary venous internal volume will generally correspond to the ejection phase of left ventricular systole, while the Z_{dia} corresponding to the minimum coronary venous volume will generally correspond to the left ventricular end diastolic period. Both the ejection phase of systole and the end diastolic period may be identified and time-stamped based on ECG data, and coordinated within the pulse generator 12 to time-stamped coronary vein impedance measurement values to identify Z_{sys} and Z_{dia}. In still other embodiments, sampling rates for the coronary vein impedance measurements may be coordinated based on ECG data, such that coronary vein impedance is only measured at times corresponding to end systole and end diastole. Still other techniques for estimating ΔVₘₐₓ from the measured coronary vein impedance values will be apparent to those skilled in the art based on the foregoing.

It is emphasized, however, that intermediate determination of the changes in internal coronary venous volume need not be made. That is, in various embodiments, the desired hemodynamic performance parameter values may be estimated directly from the desired systolic and diastolic impedance values Z_{sys} and Z_{dia}, and so intermediate ΔVₘₐₓ and/or LVP determinations and/or waveforms need not be generated.

Calibration of the hemodynamic performance monitoring system may be performed at implant or thereafter using any temporary invasive or non-invasive technique, e.g., echo, pulse pressure, and the like. In various embodiments, calibration may be performed using an external blood pressure cuff. In one embodiment, an external blood pressure measurement means, e.g., blood pressure cuff, may be used in combination with an advanced patient management system. By way of illustration only, and without limitation, one exemplary such system is the LATITUDE® Patient Management system provided by Boston Scientific Corporation. With such a system, a patient may routinely perform blood pressure checks using an external blood pressure cuff or other non-invasive device. The blood pressure data may then be communicated to the patient management system where it can be stored and also communicated back to the implanted pulse generator 12. Non-invasive arterial systolic and diastolic external blood pressure measurements may then be coordinated, e.g., based on time stamps, with the implanted corresponding to the above-described changes in internal volume of the chamber 180 during the cardiac cycle. Such changes in impedance, in turn, may be utilized to estimate changes in the internal volume of the chamber 180 during the cardiac cycle in substantially the same manner that the electrodes 60, 64 may be utilized to estimate changes in the internal coronary venous volume. That is, the change in internal volume of the chamber 180 corresponding to its difference between the internal volumes at systole and diastole will correspond substantially to the changes in internal volume of the coronary vein 34 itself and can be used to estimate and evaluate cardiac hemodynamic performance over time in substantially the same manner.

In various embodiments, the lead 114 may be deployed with the balloon in an undeflated state (not shown) so as to not significantly increase the profile of the lead 114. After implantation, the balloon 174 can be inflated by introducing a fluid through an inflation lumen (not shown) in fluid communication with the chamber 180. As will be understood by those skilled in the art, the inflation lumen can be sealed at its access point, so as to retain the balloon 174 in its inflated state. In various embodiments, the fluid for filling the balloon is an incompressible fluid, e.g., saline. In some embodiments, the lead 114 may include a reservoir (not shown) in fluid communication with the chamber 180, which can receive fluid displaced from the chamber 180 as the internal volume of the latter is reduced by increasing coronary venous blood pressure. In some embodiments, the balloon 174 is sized so as to provide an occlusive effect within the coronary vein 34 in substantially the same manner as the various embodiments of the occlusion member 74 described above with respect to the lead 14.

In the various embodiments described above, the coronary vein volume sensing elements are coupled directly to the respective leads. In other embodiments, such sensing elements may be integrated into structures separate from and deployable to cooperate with the leads. For example, the foregoing impedance sensing electrodes may be incorporated into a separate device deployable down a lumen of the lead. Such structures are described in connection with implantable pressure sensors in commonly assigned U.S. Pat. No. 6,666,626, which is incorporated herein by reference.

The various embodiments of the systems and methods for measuring coronary venous dimensional parameters described herein can be employed to perform a variety of diagnostic and therapeutic functions. In one embodiment, the coronary vein dimensional data is utilized to determine the effectiveness of drug and/or cardiac resynchronization therapies for patients with coronary diseases such as congestive heart failure. As such, the clinician may modify the drug dosages and/or adjust cardiac resynchronization therapy parameters based on hemodynamic performance information derived from the dimensional data. Alternatively or additionally, the coronary vein dimensional data may be used directly by the pulse generator 12 in a closed-loop cardiac resynchronization therapy system to adjust and optimize pacing parameters. Still other therapeutic uses for the coronary vein dimensional data may include controlling pacing stimuli for intermittent wall stress therapy, and rhythm discrimination for tachycardia therapy.

The coronary vein dimensional data may also be useful for detecting decompensation in congestive heart disease patients. Given the correlation between the coronary vein dimensional data and other physiologic measures such as LVP, still additional uses for the dimensional data will be apparent based on the foregoing.

In other embodiments, the hemodynamic performance monitoring systems described above may be stand-alone diagnostic systems, i.e., not integrated with a cardiac rhythm management device configured for electrically stimulating the heart.

Various modifications and additions can be made to the exemplary embodiments discussed without departing from the scope of impedance sensor recordings, and a two-point calibration process may then be performed based on these values. The calibration process may be repeated whenever the patient captures new blood pressure recordings. Of course, other embodiments may utilize different calibration techniques.

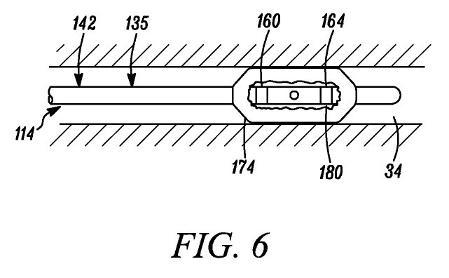

FIG. 6 is a schematic partial cut-away view of a lead 114 positioned in the coronary vein 34 according to another embodiment of the present invention. As shown in FIG. 6, the lead 114 is configured overall similarly to the leads 14 in FIGS. 2 and 3, and includes a lead body 135, a distal end portion 142, a pair of electrodes 160, 164 coupled to the lead body 135 in the distal end portion 142, and an inflatable balloon 174 coupled to and, when inflated, extending radially from the exterior surface of the lead body 135 to form an interior chamber 180. In the embodiment shown in FIG. 6, the electrodes 160, 164 are positioned within the chamber 180, which is also filled with a fluid, e.g., saline. The electrodes 160, 164 are configured to sense impedance within the chamber 180 in the manner described above with respect to the electrodes 60, 64 of the lead 14.

In the illustrated embodiment, the inflated balloon 174 is positioned in the coronary vein 34 such that blood pressure therein tends to impart a compressive hydrostatic force on the balloon 174 and thereby affect the interior volume of the chamber 180. In various embodiments, the balloon 174 is configured to be sufficiently compliant so as to permit the interior volume of the chamber 180 to fluctuate with the normal fluctuations in coronary venous pressure during the cardiac cycle. That is, the interior volume of the chamber 180 will tend to be reduced as coronary venous pressure increases during systole, and the interior volume of the chamber 180 will tend to increase as coronary venous pressure drops during diastole.

As can be seen in FIG. 6, the electrodes 160, 164 are positioned to sense changes in impedance within the chamber 180 the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method of estimating cardiac hemodynamic performance, the method comprising:
generating a signal indicative of at least one dimensional parameter of a coronary vein of a patient's heart using a sensing element on a medical electrical lead, the sensing element positioned within the coronary vein;
estimating a hemodynamic performance parameter as a function of changes in the signal during each of a plurality of cardiac cycles.

2. The method of claim 1 wherein generating the signal indicative of at least one dimensional parameter includes generating a signal indicative of an internal volume of a portion of the coronary vein.

3. The method of claim 1 wherein generating the signal indicative of the internal volume of the portion of the coronary vein includes generating a signal representing localized electrical impedance within the portion of the coronary vein.

4. The method of claim 3 wherein the medical electrical lead has a pair of longitudinally spaced electrodes, the pair of electrodes forming at least a portion of the sensing element.

5. The method of claim 4 wherein generating the signal representing the localized electrical impedance within the portion of the coronary vein includes sending an electric current to one of the pair of electrodes and sensing a corresponding electric field at the other electrode.

6. The method of claim 5 wherein estimating the hemodynamic performance parameter includes subtracting, for each cardiac cycle, a first value of the signal from a second value of the signal, the first value corresponding to an end diastolic period of the cardiac cycle and the second value corresponding to an ejection phase of a systolic period of the cardiac cycle.

7. A method of estimating cardiac hemodynamic performance, the method comprising:
estimating a change in a dimensional parameter of a coronary vein during a cardiac cycle;
estimating a hemodynamic performance parameter based on the change in the dimensional parameter during each of a plurality of cardiac cycles.

8. The method of claim 7 wherein estimating the change in the dimensional parameter includes estimating a change in volume of a portion of the coronary vein during the cardiac cycle.

9. The method of claim 8 wherein estimating the change in volume of the coronary vein includes estimating a change in localized impedance within the portion of the coronary vein during the cardiac cycle.

10. The method of claim 8 wherein estimating the change in volume of the coronary vein includes estimating a difference between a maximum volume and a minimum volume of the portion of the coronary vein during the cardiac cycle.

11. The method of claim 10 wherein the maximum volume of the portion of the coronary vein is measured at an ejection phase of a systolic period of the cardiac cycle, and wherein the minimum volume of the portion of the coronary vein is measured at an end diastolic period of the cardiac cycle.

12. The method of claim 7 wherein estimating the hemodynamic performance parameter includes:
generating a waveform indicating the change in the dimensional parameter during each of the plurality of cardiac cycles;
estimating changes in left ventricular pressure over the plurality of cardiac cycles based on the waveform; and
estimating the hemodynamic performance parameter based on the estimated changes in left ventricular pressure.

13. A method of treating a heart of a patient, the method comprising:
generating a signal indicative of a dimensional parameter of a coronary vein;
estimating a hemodynamic performance parameter as a function of changes in the signal during each of a plurality of cardiac cycles; and
selecting a therapeutic response based at least in part on the cardiac hemodynamic performance parameter.

14. The method of claim 13 wherein selecting the therapeutic response includes selecting a pacing rate.

15. The method of claim 13 wherein selecting the therapeutic response includes adjusting a drug therapy.

16. The method of claim 13 wherein selecting the therapeutic response includes turning pacing therapy on and off for cardiac wall stress adjustment.

17. The method of claim 13 wherein selecting the therapeutic response includes tachycardia therapy discrimination.

18. A system for estimating hemodynamic performance of a heart, the system comprising:
a medical electrical lead configured to be partially implanted in a coronary vein of the heart, the lead including a sensing element configured to generate a signal indicative of a dimensional parameter of the coronary vein; and
a processor operatively coupled to the lead configured to estimate a hemodynamic performance parameter based on changes in the signal during each of a plurality of cardiac cycles.

19. The system of claim 18 wherein the dimensional parameter is an internal volume of a portion of the coronary vein.

20. The system of claim 19 wherein the signal is a localized impedance within a portion of the coronary vein.

21. The system of claim 18 wherein the lead includes a plurality of longitudinally spaced electrodes configured to sense localized impedance in the portion of the coronary vein and to generate the signal therefrom.

22. The system of claim 21 wherein the electrodes have a spacing therebetween of from about 0.5 centimeters to about 2 centimeters.

23. The system of claim 18 wherein:
the lead includes:
an inflatable member configured to radially expand upon introduction of an inflation fluid therein to define a chamber; and
a plurality of electrodes disposed on the lead and configured to sense localized impedance in chamber; and
the signal indicative of the dimensional parameter of the coronary vein is a signal indicative of the localized impedance in the chamber.
